(19) United States
(12) Patent Application Publication
(10) Pub. No.: US 2022/0401285 A1
(43) Pub. Date: Dec. 22, 2022

(54) DEVICE FOR ASSISTING MOTION OF A JOINT

(71) Applicant: President and Fellows of Harvard College, Cambridge, MA (US)

(72) Inventors: Patrick Murphy, Cambridge, MA (US); Brendan Thomas Quinlivan, Cambridge, MA (US); David Christopher Perry, Cambridge, MA (US); Asa M. Eckert-Erdheim, Cambridge, MA (US); Dorothy Cecile Orzel, Cambridge, MA (US); Taylor Beth Greenberg-Goldy, Cambridge, MA (US); Conor J. Walsh, Cambridge, MA (US)

(73) Assignee: President and Fellows of Harvard College, Cambridge, MA (US)

(21) Appl. No.: 17/777,972

(22) PCT Filed: Nov. 17, 2020
(86) PCT No.: PCT/US2020/060825

§ 371 Date: May 18, 2022

Related U.S. Application Data
(60) Provisional application No. 62/937,301, filed on Nov. 19, 2019.

(51) Int. Cl. A61H 3/00; A61H 1/02

(52) U.S. Cl. CPC A61H 3/00; A61H 1/024; A61H 1/0244; A61H 1/0266; A61H 2201/1215; A61H 2201/149; A61H 2201/165

(57) ABSTRACT

In some embodiments, a device for assisting motion of a joint may include a first anchor on a first side of the joint, a second anchor on a second side of the joint, a spring operatively coupled to the first anchor and the second anchor, and an actuator operatively coupled to the first anchor and the second anchor. Actuating the actuator may apply a torque about the joint that is resisted by a reaction torque applied to the joint by the spring.

### CROSS-REFERENCE TO RELATED APPLICATIONS

This application claims the benefit under 35 U.S.C. § 119(e) of U.S. provisional application Ser. No. 62/937,301, filed Nov. 19, 2019, the disclosure of which is incorporated by reference in its entirety.

### GOVERNMENT LICENSE RIGHTS

This invention was made with government support under HD088619 awarded by National Institutes of Health. The government has certain rights in the invention.

### FIELD

Disclosed embodiments are related to devices for assisting motion of a joint.

### BACKGROUND

Both rigid and soft robotic exosuits have been used for assisting human motion in both healthy and ailing populations. For example, previous soft exosuits focusing on the lower limb(s) of a user have used Bowden cable actuators which allow the user to carry the majority of the system mass at the torso while providing assistive forces distally at the hip, knee and/or ankle.

### SUMMARY

In one embodiment, a device for assisting motion of a joint includes a motor, a mandrel operatively coupled to the motor, a tether operatively coupled to the mandrel and configured to wind onto the mandrel when the mandrel is rotated by the motor, a guide configured to guide the tether as it is wound onto the mandrel, and at least one spring. The guide is configured to move in a direction substantially parallel to an axial length of the mandrel. The at least one spring is configured to bias the guide to a neutral position along the length of the mandrel.

In another embodiment, an actuator includes a tether and a tether enclosure configured to at least partially enclose at least a portion of the tether, where the tether enclosure is configured to extend and/or retract when the tether extends and/or retracts from the actuator.

In yet another embodiment, a device for assisting motion of a joint includes a first anchor configured to be attached to a first body portion on a first side of the joint, a second anchor configured to be attached to a second body portion on a second side of the joint, an actuator operatively coupled to the first anchor and the second anchor, and a support operatively coupled to the first anchor and the second anchor. The support is also configured to substantially maintain a position of the first anchor relative to the first body portion and/or a position of the second anchor relative to the second body portion. The support is configured to apply a torque to the joint when motion of the joint deforms the support.

In still another embodiment, a device for assisting motion of a joint includes a first anchor configured to be attached to a first body portion on a first side of the joint, a second anchor configured to be attached to a second body portion on a second side of the joint, a spring operatively coupled to the first anchor and the second anchor, and an actuator operatively coupled to the first anchor and the second anchor. Actuating the actuator applies a torque about the joint that is resisted by a reaction torque from the spring.

In another embodiment, a method of assisting motion of a joint includes applying a first torque to the joint in a first direction with an actuator, and applying a second torque to the joint in a second direction with a spring that is antagonistic to the first torque.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A is a side view of one embodiment of a device for assisting motion of a joint;

FIG. 1B is a side view of another embodiment of a device for assisting motion of a joint;

FIG. 1C is a side view of another embodiment of a device for assisting motion of a joint;

FIG. 1D is a side view of another embodiment of a device for assisting motion of a joint;

FIG. 1E is a side view of another embodiment of a device for assisting motion of a joint;

FIG. 2A illustrates a device for assisting motion of a joint without support;

FIG. 2B illustrates a device for assisting motion of a joint with a rigid support;

FIG. 2C illustrates a device for assisting motion of a joint with a compliant support;

FIG. 3 illustrates one embodiment of an interface between a support 114 and an anchor 105;

FIG. 4A is a side view of one embodiment of a device for assisting motion of a joint disposed on a user's knee;

FIG. 4B is a side view of one embodiment of device for assisting motion of a joint disposed on a user's hip;

FIG. 5A is a side view of one embodiment of a device configured to assist motion of a user's hip;

FIG. 5B is a side view of one embodiment of a device configured to assist motion of a user's ankle in dorsiflexion;

FIG. 5C is a schematic of one embodiment of a device configured to assist motion of a user's ankle in plantarflexion;

FIG. 6 depicts one embodiment of an actuation module with a motor coupled to a mandrel;

FIG. 7 depicts one embodiment of a portion of an actuation module with a guide;

FIG. 8A is a schematic of the operating principle of a guide;

FIG. 8B is a schematic comparing different types of guides in different loading conditions;

FIG. 9A depicts one embodiment of an actuation module;

FIG. 9B is a cut-away view of a portion of an actuation module;

FIG. 10 depicts one embodiment of a modular actuator;

FIG. 11 depicts some benefits of a tether enclosure of an actuation module;

FIG. 12 depicts one embodiment of actuation modules integrated into a wearable system;

FIG. 13A is a graph of actuator torque output during zero torque control; and

FIG. 13B is a graph of actuator torque output during assistive torque control.

### DETAILED DESCRIPTION

As described above, previous soft exosuits have used Bowden cables to control actuation of the user's joints. However, Bowden cables often use stiff steel rope, which may be heavy, may require powerful motors to manipulate, and/or may have a low bend radius. While such an actuation strategy may be appropriate in certain applications, heavy tethers and large actuators may be undesirable in other applications, such as with users who may be impaired or otherwise unable to carry heavy components.

The Inventors have appreciated that a single unidirectional actuator may be combined with a spring to assist motion of a joint in two directions. For example, an actuator may exert a torque on a joint in one direction, while a spring may exert an antagonistic torque that opposes the torque applied by the actuator. Whereas before two actuators may have been used in an exosuit to achieve two degrees of freedom, the combination of an actuator and an antagonistic spring may allow a single actuator to achieve similar functionality, potentially yielding a smaller, lighter, less expensive, and less complex system. Such an arrangement may be particularly advantageous in certain populations of users. For example, stroke patients may benefit from a light system that applies a constant toe-up assistance to an ankle joint. However, systems used for assisting the motion of other joints are also contemplated as described further below.

In some embodiments, a device for assisting motion of a joint may be configured to provide assistance to a user's ankle. The device may comprise an actuator combined with a spring. Throughout the gait cycle, the spring may provide a dorsiflexion (toe up) torque to the ankle. When push-off assistance is needed, the actuator, located on the user's calf, may be activated to exert a plantarflexion (toe down) torque that overcomes the torque of the spring and delivers a net plantarflexion torque to the ankle. When push-off assistance is no longer needed, the actuator may be deactivated, enabling the dorsiflexion torque from the spring to provide a net dorsiflexion torque to the ankle. Such a configuration may yield a favorable failure mode, such that if the actuator fails, the user's ankle will default into a dorsiflexed state. However, in other embodiments, the torques provided by the spring and the actuator may be reversed, such that the spring provides a plantarflexion torque and the actuator applies a dorsiflexion torque. Additionally, depending on the particular mode of operation, the actuator torque applied to a joint may be controlled relative to the torque applied to the joint by an associated spring to apply a desired net torque at any point within a movement cycle of the joint as elaborated on further below.

It should be understood that the springs discussed herein relative to the various disclosed embodiments may correspond to any appropriate type of spring or compliant structure capable of applying the desired torque and/or force in a desired direction to an associated joint. For example, appropriate types of springs may include, but are not limited to, one or more selected from the group of: a tension spring, a compression spring, a torsional spring, a leaf spring, a compliant elongated structure such as a tube, rod, shaft, or other appropriate structure which may including one or more curves along its length, and/or any other compliant structure that is configured to provide the desired operating properties to apply a torque to a joint. In some embodiments, a spring may have a rotational stiffness about a joint between about 0.15 Nm/° and 0.6 Nm/°. For example, a spring may have a rotational stiffness of 0.3 Nm/°. Of course, it should be understood that a spring may have any appropriate range of rotational stiffness including ranges both less than and greater than those noted above as the disclosure is not limited in this fashion.

The actuators described herein may include any appropriate type of motor. For example, an actuator may include a brushed DC motor, a brushless DC motor, a stepper motor, and/or any other appropriate type of motor. In some embodiments, an actuator may be a linear actuator, such as a solenoid, a McKibben actuator, or a leadscrew. In some embodiments, an actuator may be directly connected to both anchors, a tether may extend from the actuator to one anchor, the actuator may be connected to anchors located on opposing sides of a joint via two or more separate tethers, and/or any other appropriate arrangement for connecting the actuator to the associated portions of a device may be used. In some embodiments, an actuator may be removable from an assistive device. If the device includes a spring, removing the actuator may yield a passive system that may provide joint assistance in only a single direction using the spring. Additional discussion of actuators, associated springs, and overall devices is provided in further detail below.

For the sake of clarity a majority of the embodiments described herein are directed to a device used to apply an assistive torque to an ankle joint. However, should be understood that the systems and methods described herein are not limited to use only with ankle joints. For example, the actuators, devices, and methods described herein are generally applicable for applying assistive torques to any joint about which it is desirable to apply an assistive torque during a motion cycle, which includes, for example, a gait cycle. This may include, but is not limited to, joints such as a knee, hip, ankle, wrist, elbow, shoulder, back, or any other suitable joint. Accordingly, it should also be understood that the anchors of a device may be attached to any appropriate portion of a person body including, but not limited to, a foot, calf, thigh, waist, torso, shoulder, back, upper arm, forearm, hand, neck, head, and/or any other appropriate portion.

In addition to the various portions of a body a device may be used to apply assistive torques to, a device may include any appropriate type of anchors for maintaining a position and/or orientation of a portion of a device relative to an underlying portion of a user's body. For example, an anchor may include cuffs, straps, flexible garments such as compression sleeves, inextensible garments, semi-rigid shells and/or rigid shells contoured to an underlying body portion of a user, and/or any other appropriate structure capable of positioning and maintaining a portion of a device on a desired portion of a user's body. For example, an anchor on the upper calf of a user may be made of a substantially inextensible garment material shaped to conform to the contours of the user's calf when worn.

In some applications, device comfort during operation may impact the duration for which a user may be willing to use the device. For example, in a soft exosuit, the portions of the device positioned on the underlying tissue may be supported entirely by shear forces on the human body. This loading may lead to discomfort and may result in the wearable component's position on a user's body drifting during use where the underlying body anatomy is not shaped appropriately to oppose this motion. Thus, when larger assistive forces are used, increased drifting of the system components may occur. Accordingly, a fully soft architecture may not be ideal for prolonged wear in some applications. Rather, a device may include one or more supports that may be employed to reduce the shear loading applied to the underlying portions of a user's body. For example, one or more supports may be attached to and extend between two anchors of a device positioned on either side of a joint. The one or more supports apply a force to the two anchors that acts to substantially maintain the two anchors in a spaced apart configuration. In other words, the support may apply a forces to each anchor that includes a component directed away from the other anchor which may help to hold the anchors in place and offset at least a portion of the shear load applied to the underlying portions of a user's body. It should be understood that while the relative position and/or orientation of the anchors may be substantially maintained relative to one another some minor shifting between the anchors may occur but the overall general position and/or orientation may be maintained. Depending on the particular embodiment, and as elaborated on further below, the one or more supports may either be one or more rigid supports and/or one or more compliant supports that are capable of deforming during use may be used. For example, a rigid support may include one or more rotatably coupled segments connected using any appropriate coupling such as a hinge, pin joint, or similar arrangement. Alternatively, a compliant structure that is capable of deforming during movement of a joint while providing a desired axial stiffness and/or rotational stiffness may be used. Specific examples of these structures are provided in more detail below.

In view of the above, in some embodiments, a device includes a first anchor configured to be attached to a first body portion on a first side of the joint and a second anchor configured to be attached to a second body portion on a second side of the joint. An actuator is operatively coupled to the first anchor and the second anchor. A support is operatively coupled to the first anchor and the second anchor. In some embodiments, the support is compliant such that it may be viewed as a spring. Alternatively, the device may include a spring that is separate and apart from the support. In those embodiments in which the support is a spring, the spring comprises one or more selected from the group of a tension spring, a torsional spring, a leaf spring, and a curved elongated structure. In some embodiments, the support is substantially aligned with and/or extends in a direction substantially parallel with an axial direction of a limb segment associated with the support in at least a portion of a motion cycle. In either case, during operation, the support may help maintain a spacing and/or orientation of the first anchor and the second anchor relative to each other and/or the underlying portion of a body part associated with each anchor during mode of operation.

In some embodiments, two or more supports may be disposed on opposing sides of a user's joint such that the supports do not impede motion of the joint. For example, in an actuation module that is configured to assist a user's ankle, two supports may be included: one on the lateral side of the ankle, and one on the medial side of the ankle. The two supports may be interchangeable, or each support may be specifically designed for a single side of a joint. In some embodiments, a support may be configured to have a multi-dimensional shape, and may wrap around the front or back of a user's limb segment and/or joint. Accordingly, it should be understood that a support for a joint may have a number of different configurations and may be positioned on any number of different sides of a joint depending on the particular design.

In one embodiment, a support may be a curved elongated structure made from a compliant material. Such an embodiment may provide a wide range of motion and may not need precise alignment with an axis of rotation of a joint as may be needed with rigid structures or certain types of springs. When loaded by an actuator, the geometry and material properties of the curved elongated structures may yield low stiffness when considering rotation of a user's foot/other joint, but may yield relatively high stiffness when considering vertical translation of an anchor at the user's calf, which may result in a significant reduction in shear loading at the user's body.

In some embodiments of a curved elongated structure, the elongated structure may have a cross section with a diameter, or other transverse dimension, between about 0.25 inches and 0.5 inches including, for example 0.375 inches. The elongated structure may also include a curve along at least a majority of its length, and in some instances along substantially its entire length. The curve may have a radius of curvature between about 20 cm and 40 cm, though instances in which multiple curves with similar or different radii of curvature are used are also possible. The length of the compliant support, which may also be viewed as a spring, may be any length suitable for the body dimensions of a given user. However, in embodiments in which the support is used for applying a torque about an ankle of a user, the support may have a distance between its two opposing ends that is between or equal to 32 cm and 53 cm, depending on the body dimensions of a user. However, it should be understood that the stiffness and other properties of a support are dependent on both the material properties and overall geometry of the support. Accordingly, depending on the material used, it should be understood that different dimensions both greater and less than those noted above may be used as long as the support provides a desired rotational and compressive stiffness.

As noted above, in some embodiments, a support may apply forces to maintain the anchors of a device in a desired position and/or orientation relative to each other when worn by a user. Accordingly, an appropriate stiffness for providing this force may be selected. In some embodiments, a support may have a linear stiffness of at least 6 N/mm, 8 N/mm, 10 N/mm, or any other appropriate stiffness. Correspondingly, a support may have a linear stiffness less than about 15 N/mm, 10 N/mm, and/or any other appropriate stiffness. Combinations of the foregoing are contemplated including for example a support with a linear stiffness between or equal to 6 N/mm and 15 N/mm or 8 N/mm and 10 N/mm. Of course depending on a particular application linear stiffnesses both greater and less than those noted above are contemplated.

A support, including a compliant support, may be made from any appropriate material with a desired combination of elasticity and yield strength for a given application. For example, in one embodiment a support may be made from a polyether ether ketone (PEEK) though any appropriate plastics, metal, composite material (e.g. polymer fiber composites and other composites), and/or any other appropriate material may be used to form a support as the disclosure is not limited in this manner.

In some embodiments, a compliant support that is configured to act as a spring to apply a torque around an associated joint may be configured such that it deforms along an expected path of motion of an associated body part during joint articulation. For example, the compliant support may include one or more curves as noted above. However, the one or more curves may be selected such that as the compliant support is deformed during use to apply a torque to the associated joint an end of the compliant support follows a natural path of motion of the body portion it is attached to around the joint. In one such embodiment, a top portion of a compliant support may be held stationary at a position on a user's calf and a bottom portion of the compliant support may be attached to a shoe, insole, supporting plate, or other structure attached to a foot of a user such that the bottom portion of the compliant support attached to the foot follows the path of motion of the foot as it is deformed and applies a desired torque to the ankle joint. Of course it should be understood that similar functionality may be applied for any number of other joints as the disclosure is not limited to only ankle joints.

In some embodiments, a joint-agnostic actuator of a device for assisting motion of a joint may include a motor, a mandrel, and a tether. The motor may be operatively coupled with the mandrel in any appropriate manner to selectively rotate the mandrel in either one or both directions. Depending on the particular embodiment, the motor may be coupled to the mandrel either by a direct connection, a low gear ratio transmission, and/or any other appropriate transmission as the disclosure is not limited in this fashion. When rotated in a first direction, the mandrel's rotation may cause the tether to spool along the mandrel. In some instances, it may be advantageous for the tether to wind in a single layer around the mandrel to avoid inconsistencies in force associated with changes in radius from the tether being wound in multiple layers and/or to reduce wear on the tether during use. Thus, in some embodiments, an actuator may include a guide that is configured to guide the tether as it winds around the mandrel to ensure the tether is wound in a single layer on the mandrel as detailed further below.

In some embodiments, an actuation module may include an actuator and associated tether enclosure. The tether enclosure may be an elastic material, such as an elastic textile, or other material that surrounds at least a portion of the tether extending away from a portion of the actuator the tether enclosure is attached to. Thus, depending on the particular construction, the tether enclosure of the actuation module may protect a user from abrasion and tangling with the tether, may protect the inside of the actuator from environmental contaminants, and/or may allow the tether to be coated with a wet lubricant due to the enclosure helping to isolate the tether from the user which can significantly improve tether lifetime.

As used herein a tether may refer to any flexible elongated structure capable of being spooled into and out of an actuator for applying a tension force to a desired portion of the devices described herein. For examples, a tether may include wire ropes as may be seen in Bowden cables, braided synthetic or natural ropes, flexible flat straps, and/or any other appropriate flexible structure. Accordingly, the above noted and other appropriate types of tethers may be used with any of the embodiments described herein. However, in some embodiments, such as embodiments where smaller sized mandrels are used, a more flexible tether as compared to the relatively stiff wire ropes used in Bowden cables may be used. For instance, a flexible braided rope, or other similarly flexible tether, may be used because they are capable of accommodating a tighter bend radius without experiencing accelerated fatigue and failure in addition to the reduced motor torques associated with spooling a relative flexible tether onto a smaller diameter mandrel or other spooling structure. This may accordingly permit the use of actuators with reduced size, weight, and cost. However, it should be understood that the currently disclosed devices are not limited to any particular type of tether.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

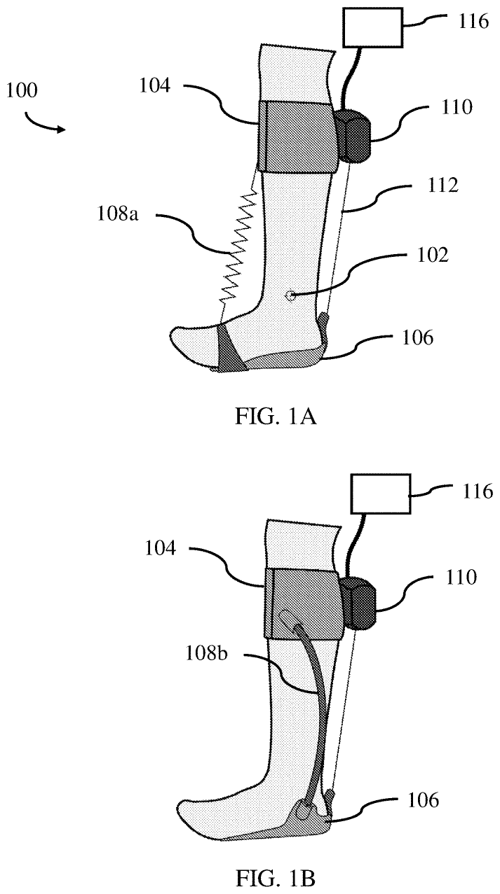

FIGS. 1A-1E show different embodiments of a device 100 configured to assist motion of a joint 102. In the embodiments shown, joint 102 is an ankle joint, although other joints are also contemplated, as elaborated on below. In the depicted embodiment, the device includes a first anchor 104 configured to be attached to a first body portion on a first side of the joint 102 and a second anchor 106 configured to be attached to a second body portion on a second side of the joint 102. For example, in the depicted embodiment, a cuff encircles a portion of a user's calf above the user's ankle and a foot plate that is constructed to receive at least a portion of a user's foot disposed thereon during use is positioned on an opposing side of the user's ankle joint. However, it should be understood that any appropriate types of anchors for maintaining a position of the device relative to the ankle and applying the desired torques to the ankle may be used. In the depicted embodiment, a spring 108 is operatively coupled to the first anchor 104 and the second anchor 106. Additionally, an actuator 110 is operatively coupled to the first anchor 104 and the second anchor 106. During use, actuating the actuator 110 applies a torque about the joint 102 in a first direction. Correspondingly, the spring may apply a torque to the joint in a second direction that opposes the torque applied to the joint by the actuator during at least one operating mode.

As noted above, in FIGS. 1A-1E, the first anchor 104 is attached to the upper calf of a user, and the second anchor 106 is attached to the foot. As noted above, the disclosed devices are not limited to any particular type of anchor for attaching the various portions of the device to an underlying body portion of a user. For example, the foot anchor may be an insole of a shoe, a strap that goes around a portion of a user's foot, an entire shoe, a plate, or any other suitable structure that functions to anchor that portion of the device in a desired position relative to the associated joint. Further, while a cuff on the calf has been depicted for the other anchor in the device, any other appropriate type of anchor for attaching the device to the user's calf, or other portion of the body, may be used as well.

In addition to the above, it should be understood that a spring used with the disclosed devices may correspond to any compliant structure capable of applying a desired force to a user's joint upon extension and/or compression. For example, as noted previously, appropriate types of springs may include, but are not limited to, one or more selected from the group of a tension spring, a torsional spring, a leaf spring, and a curved elongated structure. Exemplary embodiments of springs and their arrangement in a device are described in further detail below.

FIG. 1A depicts a spring 108a as a tension spring that is attached to and extends between portions of the first and second anchors 104 and 106 located on a front surface of the user's leg and foot to apply a tension force that biases the toes towards a toe up position (i.e. dorsiflexion). Correspondingly, the actuator 110 is operatively coupled to opposing portions of the first and second anchors located on a rear or opposing surface of the limb relative to the spring. In the specific embodiment shown in the figure, the actuator is disposed on and attached to the first anchor on the calf of the user and the actuator is attached to the second anchor by a tether 112 that extends outwards from the actuator and is attached to the second anchor such that retracting the tether with the actuator applies a torque to bias the ankle towards a toe down position (i.e. plantarflexion).

In FIG. 1B, a spring 108b included in a device corresponds to a curved elongated structure that extends between and is attached to the first and second anchors 104 and 106 along a side of the ankle joint and calf of the user, or other appropriate portion of the user's body. Similar to the above noted tension spring, the curved elongated structure may include appropriate dimensions and material properties such as curvature, cross sectional area, stiffness, and and/or any other appropriate material and/or structural property to provide a desired axial stiffness and rotational spring characteristic to provide a desired torque about the associated joint (e.g. the depicted ankle joint). Thus, the spring may also act as a support to help maintain the first and second anchors in a desired position and orientation on the corresponding portions of the body on either side of the joint. The device may include an actuator module similar to that described above relative to FIG. 1A. The figure illustrates the use of a single curved elongated structure on the outer side of the joint. However, in some embodiments, the device may include two curved elongated structures located on opposing sides of the associated limb and/or joint. For example, the device may include first and second curved elongated structures disposed on the inner and outer portions of a user's limb when worn, and which in the depicted embodiment would have been located on inner and outer portions of the user's ankle and calf when worn by the user. This may be advantageous in that the curved elongated structures may passively apply torques to the ankle that may bias the foot to a neutral position when the ankle is moved using either inversion and/or eversion movements of the ankle. Thus, the use of one or more curved elongated structures with the disclosed devices may provide torques about the ankle in one or more directions to aid movement of the ankle as well as providing an axial stabilizing force oriented along at least a portion of a length of the associated limb to maintain the two anchors in a desired spaced apart position to avoid slippage during use.

FIG. 1C depicts a device including a tension spring 108c similar to that described in FIG. 1A in addition to the noted actuator module. However, in the depicted embodiment, a rigid separate support 114 extends between and is attached to the first and second anchors 104 and 106. Specifically, first and second portions 114b and 114c of the rigid support are located on either side of a rotatable coupling 114a with the first and second portions of the rigid support attached to the first and second anchors respectively. As described above, the support may apply forces to the first and second anchors to maintain the anchors in a desired spaced apart position and orientation on the respective portions of the body located on opposing sides of the joint. To facilitate rotation of the joint, which in this embodiment is in ankle joint, the support may include the above noted rotatable coupling 114a with an axis of rotation substantially aligned with an axis of rotation of the associated joint. Accordingly, the one or more supports may apply the desired forces to the attached anchors to maintain the anchors in their desired position and orientation. Further, depending on the particular embodiment, either a single support may be used or two supports located on opposing sides of the limb may be used. The device may operate substantially similar to the device described in relation to FIG. 1A.

FIG. 1D, depicts an embodiment similar to that shown in FIG. 1C. However, in the depicted embodiment, the tension spring has been replaced by a torsion spring 108d. Specifically, the torsion attached to the first and second portions 114b and 114c of the rigid support 114 located on either side of the rotatable coupling 114a and attached to the first and second anchors 104 and 106 respectively. Thus, depending the torsion spring may apply a desired torque to the separate first and second portions of the rigid support which transmits these torques to the first and second anchors 104 and 106 attached to the first and second portions of the rigid link respectively. This torque is then transmitted to the joint aligned with an axis of rotation of the rotatable coupling through the connected portions of the user's body that works in cooperation with support 114.

FIG. 1E depicts yet another embodiment of a device for assisting movement of a joint. In the depicted embodiment, the device includes a spring 108e in the form of a leaf spring in addition to the previously discussed actuator module. The leaf spring extends between and is attached to the first and second anchors 104 and 106 located on opposing sides of the ankle joint. Depending on the stiffness of the leaf spring, the leaf spring may apply a force to the associated anchors to maintain them in a spaced apart configuration in a desired position and orientation on the corresponding portions of the body (e.g. the calf and foot). Even though the leaf spring is located on a rear portion of the calf and ankle when worn, the leaf spring may be configured and attached to the anchors such that it applies a torque in a desired direction, which in this embodiment, may correspond to a torque that biases the foot to a toe up position.

It should be understood that while the actuators and springs depicted in the above, embodiments are depicted as applying torques in various directions and are arranged on various sides of the joint and/or limb of a user, the applied torques and arrangements of these components relative to the joint and/or limb are not limited to only those shown. For example, in some embodiments, a spring may apply a torque to the joint to bias a foot to a toe down position and an associated actuator module may be operated to apply a torque to the joint to bias the foot to a toe up position. In such an embodiment, the spring may be located on a side and/or rear surface of the user's leg when worn in the actuator may be located on a front of the user's leg when worn. Accordingly, the current disclosure encompasses any number of variations regarding the specific combinations of torques and/or locations of the springs and actuators discussed herein. Additionally, the disclosure of antagonistic springs and actuators for use with assisting the movement of a joint of an individual may be applied to any appropriate joint even though the embodiments above are described relative to an ankle joint.

In addition to the relative positioning and operation of springs and actuators, FIGS. 1A-1E also depict the use of an actuator 110 that is operatively coupled to electronics 116. Electronics 116 may include a power source, one or more sensors, and/or a processor with associated non-transitory computer readable medium that includes processor executable instructions that when executed by the processor operate the device using any of the methods described herein. In some embodiments, some or all of the electronics may be integrally mounted with the actuator, though instances in which a wired and/or wireless connection between the various components of the device are used are also contemplated. In some embodiments, additional sensors may be disposed in a plurality of locations distributed throughout the actuation module and/or on other portions of the assistive device and operatively coupled processor such that corresponding sensor signals may be transmitted to and received by the processor. For example, a load cell may be placed in series with an actuator, a strain gauge may be disposed on an anchor, and/or any other appropriate type of sensor may be used in any appropriate location on the device as the disclosure is not limited in this fashion. In some embodiments, an actuator may apply a torque to a joint, and a spring and/or a support may apply a reaction torque. The reaction torque may be sensed by one or more sensors and used in a feedback control process. For example, a force and/or torque applied by an actuator may be adjusted based at least in part on a sensed reaction force and/or torque associated with a spring and/or support. A reaction force and/or torque may be sensed using force and/or torque sensors. Other sensors such as strain gauges and/or position sensors may be used to measure deformation of a spring and/or support and to calculate the reaction force and/or torque indirectly.

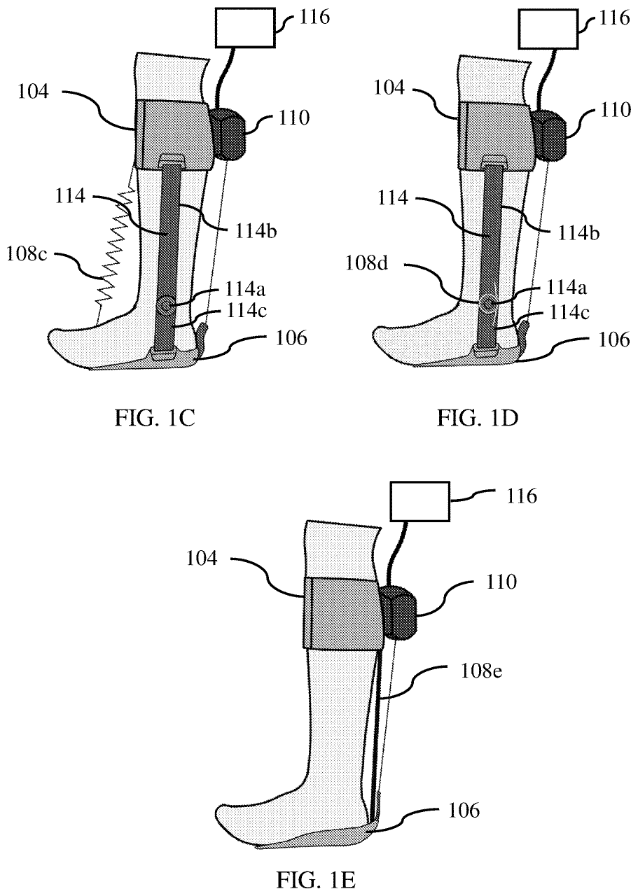

FIGS. 2A-2C illustrate the effects of providing different supports in a device for assisting motion of a joint on shear forces applied to a user's body. As described above, in some embodiments, a device for assisting motion of a joint may include a support that may apply a force to bias the associated anchors positioned on either side of a joint towards a desired position and/or to apply a torque about the joint. Again, such a support structure may reduce the shear force applied to the corresponding portions of a user's body, thereby increasing comfort and increasing the efficiency of force transfer from the actuator into joint motion. A support may also reduce slippage of anchors and generally enable better positioning of the components of the device.

FIG. 2A illustrates the effect of providing no support on shear forces applied to a user's body. With no support, the entire force of the actuator may be supported by the user's body through the application of shear forces to the underlying portions of the user's body, which in this case is the calf and foot of the user. As the actuator (or actuation module) creates tension between the two anchor points, the only structure to prevent the anchors from moving is the user's body. In such cases, the shear forces applied to a user's body may be high, and an anchor may shift significantly depending on the particular body portion they are attached to. For example, if the body portions underlying the anchors were the calf and thigh of a user, and/or if the anchor were positioned below the calf in the depicted device, the anchors would be fairly likely to drift down during use due to the natural shape of the thigh and calf relative to the applied forces making it difficult to create a fixed attachment without excessive forces being applied and/or using additional anchor structures.

FIG. 2B illustrates the effect of providing a rigid support on shear forces applied to a user's body. In cases in which the stiffness of the rigid support is much greater than the stiffness of a user's body, the force exerted by the actuator may be entirely reacted by the rigid support. In such cases, the shear forces applied to a user's body may be very small or close to zero. The shifting of anchors may be negligible in this case. However, such a rigid support may severely restrict joint motion as compared to the soft exosuit shown in FIG. 2A.

FIG. 2C illustrates the effect of providing a compliant support on shear forces applied to a user's body. A compliant support may be advantageous in that it may combine the benefits of the extreme cases (i.e., no support, rigid support) and may avoid some of the drawbacks. For example, a compliant support may reduce shear loading on the user's body (compared to no support), may not fully restrict out of plane motion (e.g., inversion and/or eversion movements of the ankle), and may function without precise alignment with an axis of rotation of a joint. A compliant support may be designed to be stiff in the vertical axis (when considering an ankle device) but to be otherwise compliant to enable natural motions for a user while also providing a desired torque about the associated joint. For example, the geometry and material properties of the support may determine in part the torque applied to the joint and the motion path of a user's body portion. In embodiments in which a compliant support is used as an antagonistic spring to an actuator, the support may have sufficient stiffness in the direction of loading from the actuator to at least partially resist the forces from the actuator. A compliant support may help to maintain separation of anchors and react loads, and may exhibit spring behavior with desired stiffness properties.

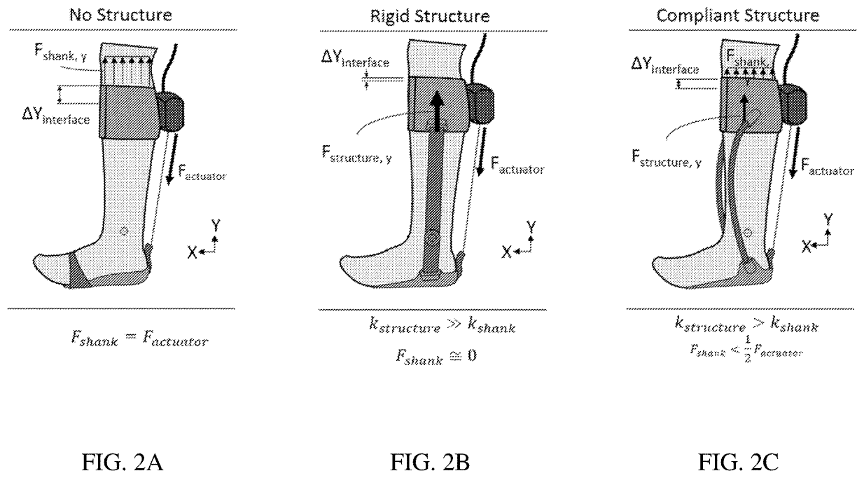

FIG. 3 illustrates one embodiment of an interface between a support 114 and an anchor 105. An end portion of the support 114 includes a support mount 122 coupled to an anchor mount 124 of the anchor 105. The support mount 122 may be coupled to the support 114 using fasteners, press fit geometry, epoxy, adhesive, or any other suitable coupling. As described above, an anchor 105 may include a worn component such as a cuff, strap, sleeve, or insole. In such embodiments, the anchor mount 124 may be sewn into the worn component to couple the anchor mount 124 to the anchor 105. However, it should be appreciated that any suitable coupling between an anchor mount 124 and an anchor 105 may be used, as the disclosure is not limited in this regard. Additionally, it should be understood that a support 114 may, in some embodiments, also be described as a spring. See for example spring 108b of FIG. 1B, which acts as a spring and as a support.

One or more sensors may be provided at the interface between a support 114 and an anchor 105. In some embodiments, a sensor 120 is disposed between the support mount 122 and the anchor mount 124. The sensor 120 may be fixedly coupled to the anchor mount 124, and the support mount 122 may attach directly to the sensor 120. That is, the support mount 122 may be directly connected to the sensor 120, and the sensor 120 may be directly connected to the anchor mount 124. The sensor 120 may be coupled to the support mount 122 and/or the anchor mount 124 using a fastener, press fit geometry, epoxy, adhesive, or any other suitable coupling. As such, in some embodiments, the support mount 122 may be indirectly coupled to the anchor mount 124 via the sensor 120. In some embodiments, the support mount 122 may be directly connected to the anchor mount 124, and the sensor 120 may be coupled to one or both of the support mount 122 and the anchor mount 124.

In some embodiments, the sensor 120 is a force sensor configured to measure forces exerted by the anchor 104 on the support 114 and/or forces exerted by the support 114 on the anchor 104. Signals from the sensor 120 may be used to calculate forces and/or torques associated with the support 114 and/or the anchor 104. Other types of sensors may be included at the interface between a support 114 and an anchor 104. For example, force and/or torque sensors may be integrated into the support 114, and/or a strain gauge 121 may be applied directly to the support 114. Alternatively or additionally, position sensors may be used to measure the deformation of the support 114. Because the support 114 may operate as a spring with a known stiffness, signals from the position sensors may be used to calculate forces and/or torques associated with the support 114. For example, a torque applied to the joint by the support 114 may be calculated using the known stiffness of the support 114 and the measured deformation of the support 114.

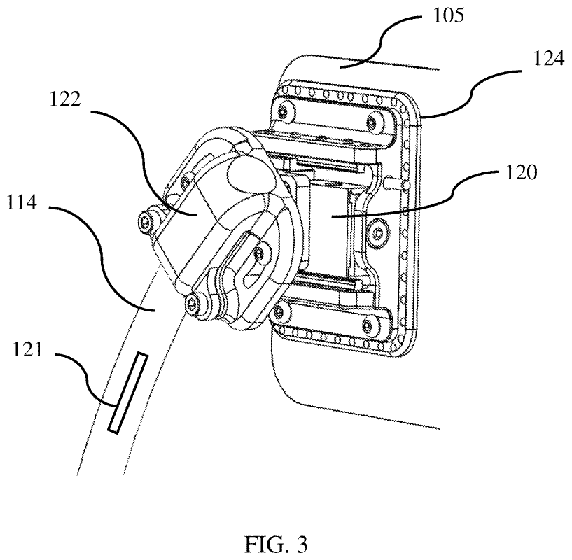

While much of the preceding description has referred to a user's ankle, calf, and foot, this is for illustrative purposes only. Accordingly, it should be understood that the device is disclosed herein may be applied to other joints and body portions for assisting motion of the corresponding joint. For example, FIGS. 4A-4B show devices disposed on other joints of a user. FIG. 4A depicts a device with anchors 104 and 106 disposed on either side of a user's knee, while FIG. 4B depicts a device with anchors 104 and 106 disposed on either side of a user's hip. In the embodiments of the figures, a compliant support 114 flexes to provide knee extension or hip flexion and the associated actuator 110 may apply antagonistic torques to provide knee flexion or hip extension. However, in other embodiments, a compliant support may flex to provide knee flexion or hip extension and actuator may provide knee extension and hip flexion. Correspondingly, a compliant support, spring, and/or an actuator may apply a torque about any joint in any desired direction. In addition to lower limb joints, an actuation module (which may include a spring, compliant support, anchors, actuator, and/or other components) may additionally be disposed on a wrist, elbow, shoulder, torso, back, or any other suitable joint.

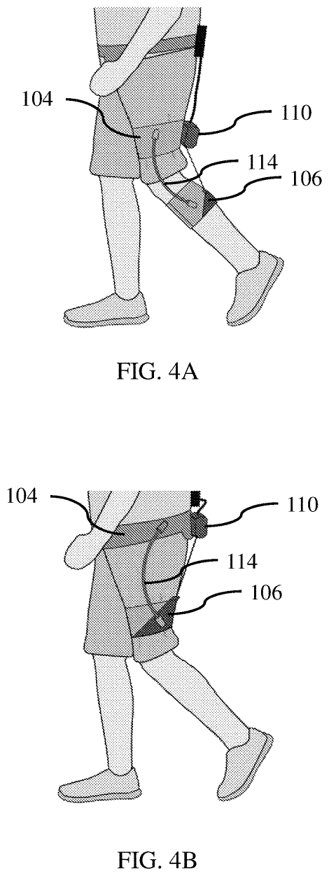

FIGS. 5A-5C depict a device configured to assist motion of a joint worn on different joints of a user, showing the modular nature of the actuation scheme. In some embodiments, an actuator may be mounted adjacent an anchor of a device, and control and power electronics may be mounted in a separate location, such as on a waist belt. Such modularity may enable a single power/control solution (e.g., electronics mounted on a waist belt) to be used with one or more joint-agnostic actuators to assist different joints (e.g., hip, knee, ankle) of a user.

FIG. 5A depicts a device configured to assist motion of a user's hip. Anchors 104 and 106 may be disposed on either side of a user's hip. For example, the first anchor 104 may be disposed above the hip, such as on a waist belt. The second anchor 106 may be disposed below the hip, such as on a portion of the thigh proximate the knee. Actuator 110 may apply a torque about the hip to assist the user's hip motion. The actuator 110 may be mounted on the first anchor 104, and may be controlled by electronics 116. The electronics may be disposed on a waist belt, or may be attached to any suitable location on or off of a user's body. FIG. 5B depicts a device configured to assist motion of a user's ankle in dorsiflexion (toe up), while FIG. 5C depicts a device configured to assist motion of a user's ankle in plantarflexion (toe down). In some embodiments, a first anchor 104 may be disposed on a user's calf, while a second anchor 106 may be disposed below the user's ankle. Second anchor 106 may be a shoe insert, a strap associated with the exterior of a shoe, or any other suitable structure. In addition to the embodiments of the figures, a device may be disposed on any user-selected joint, such as a hip, knee, ankle, or any other suitable joint, as the disclosure is not limited in this regard.

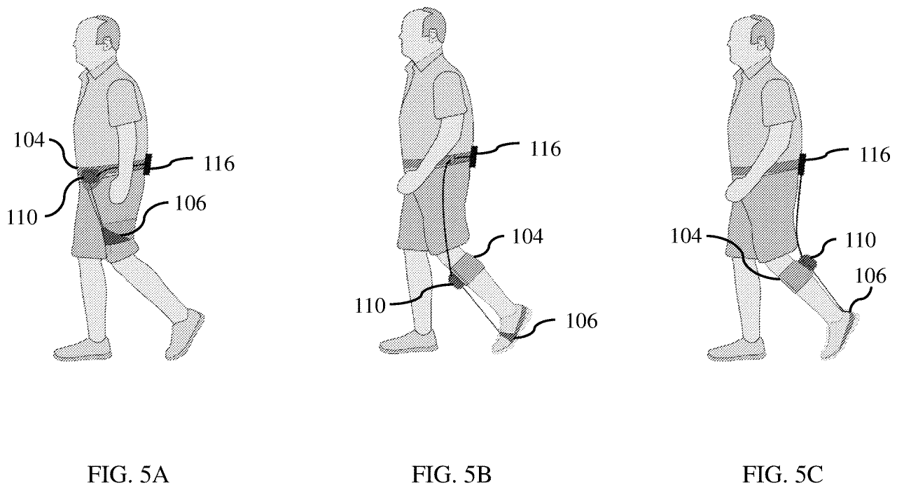

FIG. 6 depicts one embodiment of the drivetrain of an actuator in which a motor 816 is operatively coupled to a mandrel 810. The motor may be coupled to the mandrel through a belt and pulley mechanism. For example, as the output of the motor turns, a belt 820 coupled to the output of the motor 816 may turn a pulley 818 coupled to the mandrel, causing the mandrel to turn. However, other suitable arrangements of coupling a motor and a mandrel are contemplated, and the disclosure is not limited in this regard. For example, a geared, direct, and/or any other appropriate type of transmission for coupling the output of the motor to the mandrel may be used as the disclosure is not limited in this fashion. In either case, a tether 806 operatively coupled to the mandrel such that the tether may be wound around the mandrel when the mandrel is rotated by the motor. In some embodiments, the mandrel may be a cylindrical shaft. However, the mandrel may be any suitable structure with any suitable shape configured to enable winding of the tether onto the mandrel.

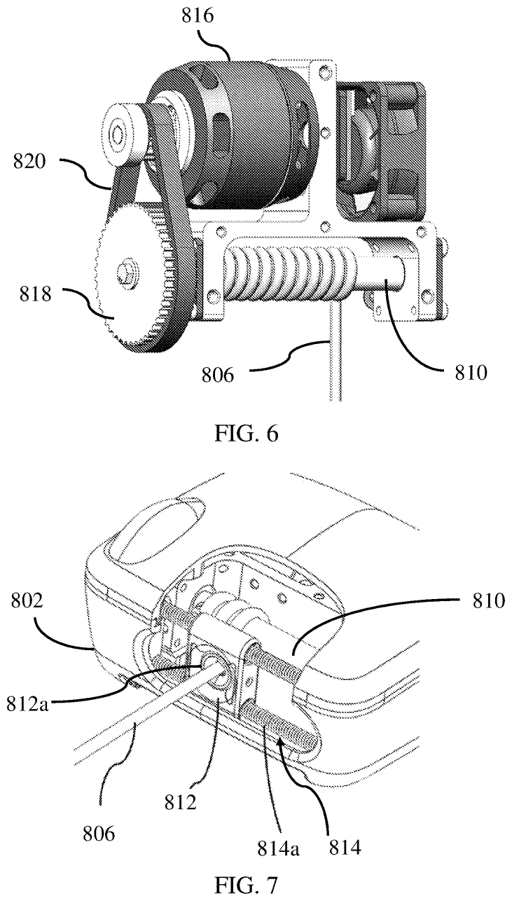

FIG. 7 depicts one embodiment of an actuator 802 including a guide 812 for controlling winding of a tether 806 onto a mandrel 810 of the actuator such that the tether may be wound onto the mandrel in substantially a single layer. For example, a guide 812 includes a tether engaging portion 812a, such a channel, through hole, or other structure that is capable of engaging with and guiding the position and/or orientation of the tether 806 as it is wound onto the mandrel 810. For example, the tether 806 may pass through a through hole formed in the guide 812 and onto the mandrel 810.

The guide 812 and/or the tether engaging portion 812a may include a low-friction material and/or coating configured to minimize wear on the tether 806. A low-friction material may also minimize a resistance to sliding motion between the tether 806 and the guide 812 and/or the tether engaging portion 812a. Non-limiting examples of low-friction materials include ceramics, plastics, and polished metals such as stainless steel, aluminum, brass, and copper with a suitably smooth surface to provide a desired low coefficient of friction for a desired application. Of course, other low-friction materials may be suitable, and the present disclosure is not limited to any specific material. In some embodiments, low-friction materials may include materials that, when in contact with a tether, are associated with a low coefficient of friction. In some embodiments, a coefficient of friction between a guide 812 (and/or a tether engaging portion 812a) and a tether 806 may be less than or equal to 0.5, 0.4, 0.3, 0.2, 0.1, or any other appropriate value. In some embodiments, a coefficient of friction between a guide 812 (and/or a tether engaging portion 812a) and a tether 806 may be greater than or equal to 0.01, 0.1, 0.2, 0.3, 0.4, or any other appropriate value. Combinations of the foregoing are also contemplated, including a coefficient of friction between a guide 812 (and/or a tether engaging portion 812a) and a tether 806 of between 0.01 and 0.5, 0.01 and 0.3, 0.01 and 0.1, and/or any other appropriate combination. Additionally or alternatively, bearings or other rolling elements may be integrated into the guide 812 and/or the tether engaging portion 812a to reduce wear on the tether 806.

In some embodiments, it may be desirable for the guide 812 to be configured to move in a direction substantially parallel to a length of the mandrel 810, such as an axial length of the mandrel. For instance, the guide may be moveably mounted on one or more rails 814 that extend in a direction parallel to an axial direction of the mandrel. In the embodiment specifically depicted in FIG. 7, the guide includes two parallel through holes that slidingly receive the two corresponding rails to maintain an orientation of the guide, and the portion of the guide engaged with the tether, relative to the mandrel while permitting the guide to move along at least a portion of the axial length of the mandrel. In some embodiments, at least one spring 814a is configured to bias the guide to a neutral position along the length of the mandrel. For example, as depicted in the figure, two or more tension and/or compression springs may be located on opposing sides of the guide to bias the guide towards a middle of the rails, or any other desired neutral position along a length of the mandrel. Further, in some instances, the springs may be disposed on the rails to help maintain their position within the actuator while providing the desired biasing forces to the guide. However, instances in which the springs are not disposed on the rails are also contemplated. Operation of such an actuator is described further below.

As stated above, it may be desirable to wind only a single layer of tether around the mandrel because a tether that winds upon itself multiple times may experience increased fatigue, and may result in a higher motor torque due to changes in the applied moment arm as the tether is wound on top of itself on a relatively small mandrel. To achieve a single layer, a winding angle θ₁ of the tether around the mandrel may be steep enough to make winding directional, but not so steep that the mandrel runs out of winding room. In some embodiments, a guide of an actuator engaged with a tether may provide a winding angle of Oi that is greater than or equal to any angle greater than 0° that ensures the rope winds in a single layer. Further the winding angle θ₁ may be less than or equal to 80°. Combinations of the above noted ranges for the winding angle are contemplated including, for example, a winding angle between or equal to 4° and 80°. Further, a guide may maintain a winding angle within any other appropriate range of angles during operation. However, maintaining such a range, or any other suitable range, of angles may be difficult when the system is mounted on a user, as the entry angle θ₂ of the tether may change constantly as a user moves about.

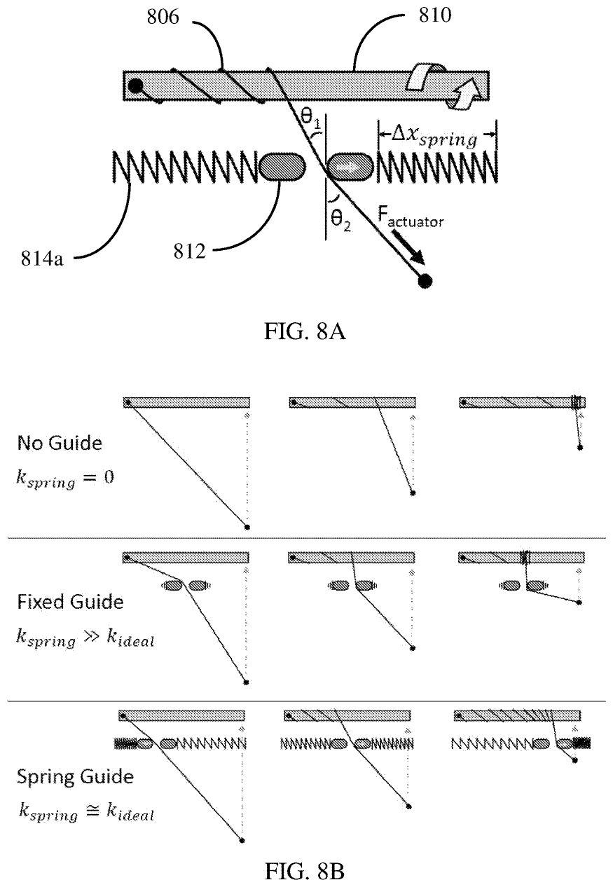

Referring to FIG. 8A, to aid in a tether winding onto a mandrel in a single layer, a guide 812 may be employed to change an extreme entry angle θ₂ of a tether 806 to be within an acceptable range of winding angles θ₁. As described previously above, the guide may include one or more springs 814 to bias the guide to a desired neutral position along a length of the mandrel, which in some embodiments is an approximately central position along a length of a portion of the mandrel along which the tether is wound during operation. As the tether spools onto the mandrel, θ₁ may decrease. This decrease in θ₁ causes a force imbalance between the two sides of the tether passing through the guide applying different forces to the guide in a direction parallel to the mandrel. The resulting force imbalance leads to the guide being displaced which compresses the springs disposed on a side of the guide corresponding to the direction of movement, and extends the springs on the opposing side of the spring. This is illustrated in the figure as an increasing change in the guide deflection (ΔX_{spring}). Thus, this movement of the guide may help to maintain an appropriate guide position, and corresponding tether position and angle of entry, for winding the tether onto the mandrel in a single layer onto the mandrel 810. As elaborated on below, the spring stiffnesses may be selected based on the expected tether entry angles and actuator forces, which may vary depending on the specific application, to provide a desired operation of the actuator.

FIG. 8B is a schematic comparing different types of guides with different types of constructions. Without wishing to be bound by theory, the location at which a tether winds on a mandrel may depend at least in part on the entry angle and winding angle of the tether regardless of the presence of a guide and the corresponding stiffness of any springs associated with the guide. For example, in the case of no guide being used, which may be approximated as a spring constant of zero, or similarly too compliant a spring, the tether simply winds onto the mandrel according to geometrical considerations until the tether is approximately perpendicular to the mandrel at which point the tether winds on top of itself. Similarly, if the spring constant of the one or more associated springs are too stiff, and/or if the guide is simply fixed in place, the tether will also wind onto the mandrel according to geometrical constraints until it is approximately perpendicular to the mandrel at which point the tether again winds on top of itself. In contrast, when an appropriate spring constant is selected, the guide is permitted to move along an axial length of the mandrel as described above. This type of operation permits a much larger amount of tether to be wound onto the mandrel without winding on top of itself for a variety of entry angles to the actuator. It should be understood that the specific geometries and spring stiffnesses will vary depending on the particular actuator geometries, tether orientations, and forces to be applied by the actuator during use.

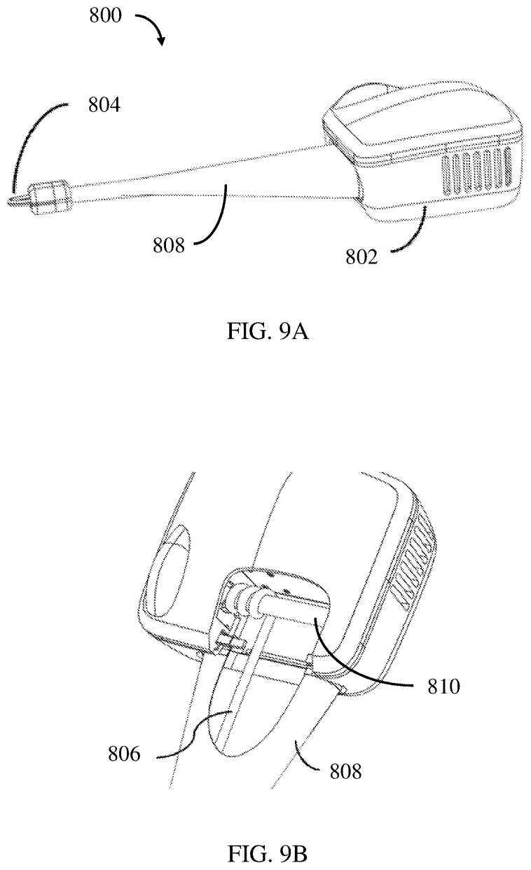

As shown in FIGS. 9A-9B, in some embodiments an actuation module 800 may include an actuator 802, a tether 806 that is wound onto a mandrel 810 of the actuator, and a tether enclosure 808 that at least partially encloses, and in some instances fully encloses the portion of the tether 806 extending out from the actuator. FIG. 9B illustrates a cut-away view of a portion of the actuation module of FIG. 9A to illustrate these internal components. In some instances, the actuation module may also include a connector 804 connected to a distal end of the tether 806 opposite the actuator. As seen in the figure, the tether enclosure is connected to and extends out from an exterior of the actuator to the associated connector in the form of a sleeve that the tether passes through, though other enclosure shapes and constructions are possible. Depending on the particular embodiment, the tether enclosure may extend up to and/or past the associated connector such that the portion of the tether extending between the connector and actuator is fully enclosed by the tether enclosure. Further, in some embodiments, a tether enclosure may taper from one end to the other. For example, a tether enclosure may be wider near an actuator housing and narrower near a distal connection point. However, some embodiments, a tether enclosure may have a uniform diameter. In certain applications, the tether enclosure is configured to selectively extend and retract as the tether extends and retracts relative to the actuator. In some embodiments, the tether enclosure is formed from an elastic material, for example an elastic textile may be used to form an elastic sleeve that encloses the tether. Though, embodiments in which the tether enclosure is not elastic are also contemplated.

The above described tether enclosures may offer a number of benefits including, but not limited to: protecting users from abrasion and tangling with an exposed tether; protecting the inside of the actuator from environmental contaminants; and allowing the tether to be coated with a wet lubricant or dry lubricant, which can significantly improve tether lifetime, without exposing the user and surrounding environment to the lubricant. In addition, and embodiments in which the enclosure is elastic, the stiffness of the enclosure may be tailored to provide a certain amount of constant tension to the connected anchors used to hold the device on corresponding portions of a user's body when the device is worn. This initial tensioning of the enclosure may be desirable for keeping components in place. For example, the stiffness of the enclosure may be selected to provide tension that holds the anchors in place while not affecting the user's kinematics. In one such application, the enclosure may exert a tensile force of about 5 N to 10 N at about 50% elongation though other ranges both greater and less than those noted above are also contemplated depending on the application. Alternatively, the stiffness may be selected in order to have a measurable impact on user kinematics (e.g., an assistive torque during a power-off state), provide a preloading tension to smooth the application of force by the mandrel, or reduce the actuator's power requirements. Of course, embodiments in which the above-noted benefits are not provided, and/or different benefits are provided, by the disclosed devices are possible as the disclosure is not limited to providing these benefits in all instances. Several of these benefits are elaborated on further below.

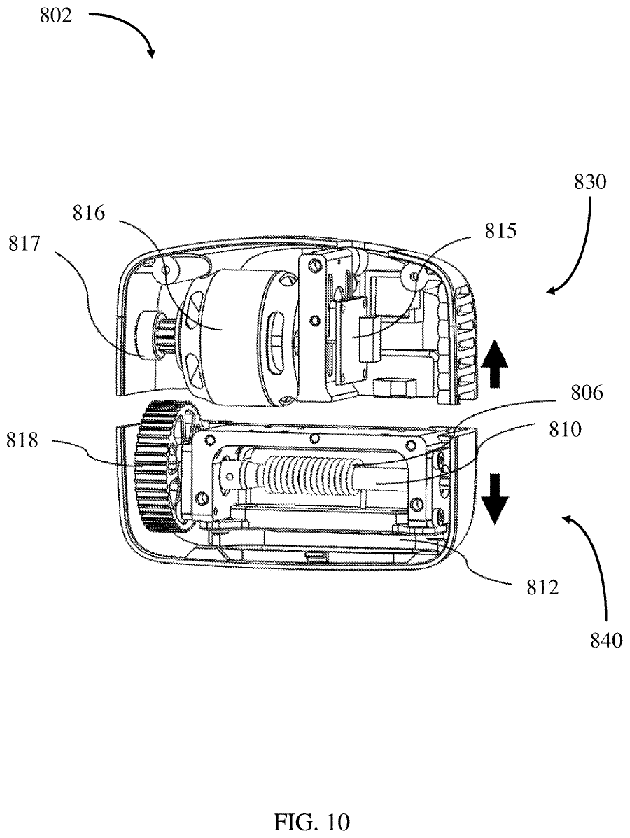

FIG. 10 depicts one embodiment of a modular actuator 802. A modular actuator may enable easy replacement of consumable components, such as high wear components. The modular actuator 802 of FIG. 10 includes a primary portion 830 and a replaceable portion 840. The replaceable portion 840 may form a single cartridge configured to selectively detach from the primary portion 830. The replaceable portion 840 may include one or more of a mandrel 810, a tether 806, a guide 812, and a first timing pulley 818. The primary portion 830 may include the remaining components of the actuator 802, such as a motor encoder 815, a motor 816, and a second timing pulley 817. Of course, it should be appreciated that in other embodiments, different combinations of components may be included in the primary portion 830 and the replaceable portion 840 of the modular actuator 802. For example, the timing pulley 818 may be included with the timing pulley 817, the motor 816, and the encoder 815 in the primary portion 830. In some embodiments, at least the mandrel 810, the tether 806, and the guide 812 are selectively removable from the motor 816. The primary portion 830 may be configured to selectively engage the replaceable portion 840 using snap- or press-fit geometries, fasteners, latches, detents, slots and protrusions, magnets, or any other suitable coupling.

In some embodiments, the primary portion 830 and the replaceable portion 840 may be engaged and/or disengaged manually. In some embodiments, components of the primary portion 830 and/or the replaceable portion 840 may aid in the engagement/disengagement of the two portions. For example, after an initial manual coupling phase of the two portions 830 and 840, motor 816 may be actuated to correlate motion of pulleys 817 and 818, which may serve as a final coupling phase to fully engage the two portions 830 and 840. In some embodiments, the primary portion and the replaceable portion may be coupled using a belt connecting two pulleys, sets of meshing gear teeth, and/or a spline coupling between two shafts. Of course, a primary portion and a replaceable portion may be appropriately coupled in other ways, and the disclosure is not limited in this regard.

A modular actuator 802 may additionally enable a user to modify the functionality of an actuation module, such as to adapt the actuation module to a particular function. For example, different replaceable portions 840 with different length tethers 806 may be selected to fit a range of user heights and sizes. Another modification would be to change the diameter of the timing pulleys 817 and 818 to change the gear ratio between the motor 816 and mandrel 810. This modification would enable a single actuation module with multiple replaceable portions 840 to be tailored to the specifications of a specific joint. For example, the ankle may require higher torque but lower speed, whereas the hip may require lower torque but higher speed. A set of replaceable portions 840 with different gear ratios could allow a single actuation module to accommodate both joints by replacing a first replaceable portion with a second replaceable portion. Changing the diameter of the mandrel 810 would have a similar effect to changing the gear ratio. Of course, other modifications may be made to an actuator 802 by replacing one replaceable portion 840 with another replaceable portion 840, and the disclosure is not limited to the modifications explicitly described herein.

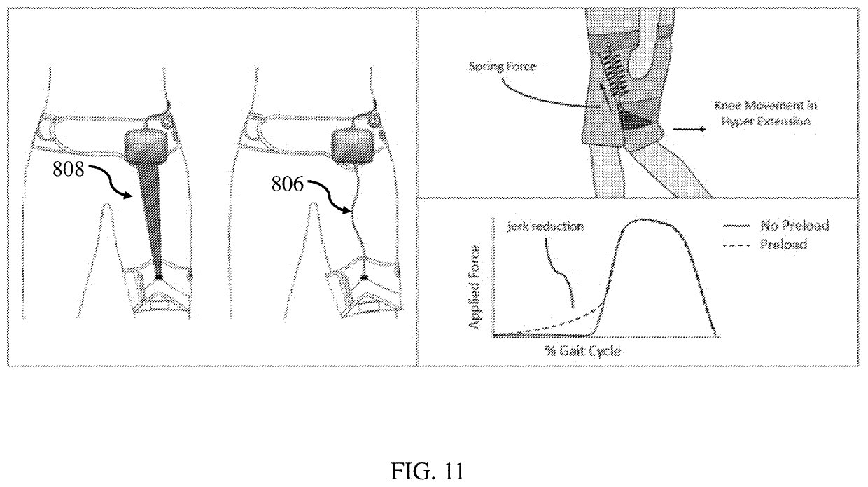

As noted above, in some embodiments, a tether enclosure may be preloaded during use such that it provides a relatively constant low tension force to the attached anchors. Again, this may help to keep the anchors in place on a user's body even while the device is unpowered. This preloading of the device can also be used to reduce actuation jerk by smoothing the transition from an unloaded to a loaded state which may provide desirable effects on kinematics such as supporting knee control by opposing motion towards hyperextension. These conditions are shown in FIG. 11, where actuators with a preloaded tether enclosure 808 and a loose unenclosed tether 806 are illustrated. As shown in the corresponding graph, the change in applied force to the associated joint is more sudden for the unenclosed tether as compared to the device including the preloaded tether enclosure which shows a more gradual smooth transition to the loaded state.

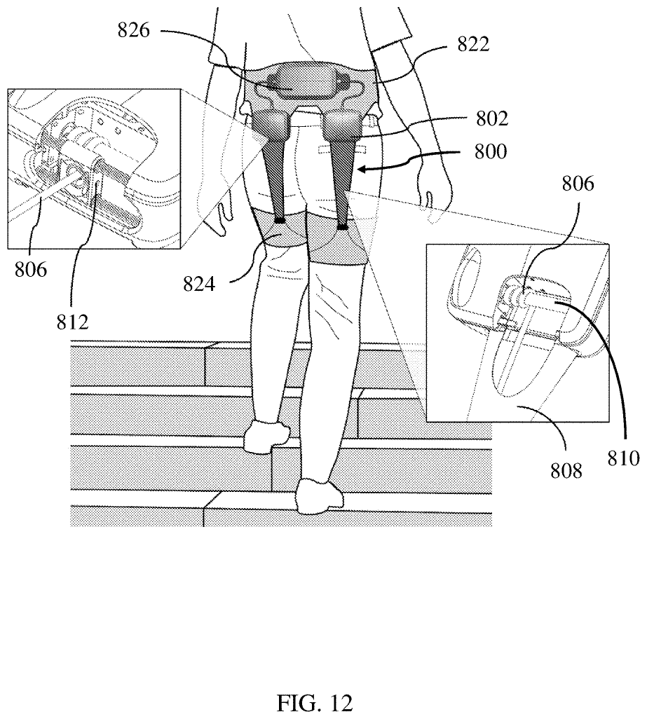

FIG. 12 depicts one embodiment of actuation modules integrated into a wearable system. In the depicted embodiment, actuation modules 800 connect a first anchor 822 to separate second anchors 824 located on the separate legs of the user. In the depicted embodiment, the first anchor 822 is a waist belt, and the second anchors 824 are wearable components worn above the knee of each leg on the thigh. As noted previously, electronics 826 may include a power source, one or more sensors, and/or a processor with associated memory used for controlling operation of the device to provide a desired combination of assistive torques to the separate hips, or other joint, of the user during a motion cycle, such as a gait cycle.

Using the disclosed actuators and antagonistic springs, it is possible to provide a wide range of torques to a joint in various orientations of the joint during a motion cycle. For example, the torques applied by the actuator may be used to allow a full torque from a spring to be applied to an associated joint, to be partially applied to the joint, to cancel out a torque applied by the spring, and/or to apply a torque that is greater in magnitude, and opposite in direction, relative to the torque applied by the spring. Thus, with the ability to control torque delivered throughout the gait cycle, various actuation profiles may be applied to a joint by a device to provide various amounts of assistive torques during the various portions of the motion cycle.

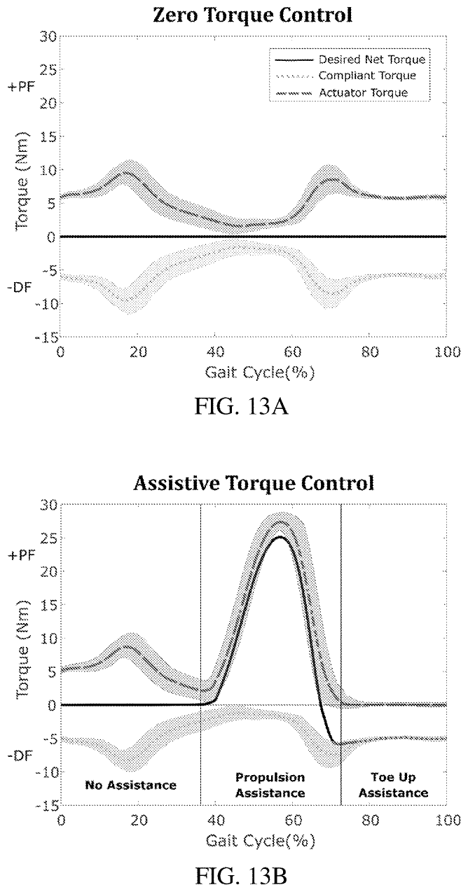

FIG. 13A is a graph of actuator torque output during zero torque control. This figure depicts a profile in which the system delivers zero net torque to the ankle during a gait cycle. For example, in the graph, an actuator applies positive torque (+PF) and a spring applies negative torque (−DF). The solid line represents the desired net torque. Throughout the gait cycle, the system monitors the torque from the spring, and delivers an actuator torque of equal and opposite magnitude to the spring which results in zero torque being delivered to the user. A zero-torque actuation profile could be useful at certain times throughout a therapy regimen in which the user wants to quickly transition between active assistance and performing exercises fully under their own power.

FIG. 13B is a graph of actuator torque output during assistive torque control. This figure depicts a scenario in which the net torque magnitude applied to the joint changes throughout the motion cycle, which in this case is the torque applied to an ankle during a gait cycle. In the graph, an actuator applies positive torque (+PF) and a spring applies negative torque (−DF). The solid line represents the desired net torque. While the foot is planted on the ground, the force from the actuator offsets the force from the spring, delivering a net zero assistive torque. As the user nears toe off, a plantarflexion torque is delivered by the system, aiding the user with propelling themselves forward. Once the user lifts their foot for swing, the actuator delivers a very low torque, allowing the spring to apply a net negative torque to the ankle assist with dorsiflexion to prevent drop foot/tripping.

In view of the above, it should be understood that the disclosed actuators and antagonistic springs may be used to apply any desired combination of positive, negative, and/or zero net torques to an associated joint during the various portions of a motion cycle without the use of antagonistic actuators. Further, such systems may provide desired failure and/or unpowered modes, where they may still provide a desirable assistance, such as torques to bias a foot towards a toe up position, even when operating in a passive unpowered or failure mode. The systems may also be operated to vary the amount of assistance provided to a user over time. For example, during initial therapy a system may provide a first amount of assistive torques during motion which may be reduced over time as a user's joint is rehabilitated and less assistance is needed. Alternatively, the system may provide assistive torques with a first magnitude during normal use and may be operated to provide lower assistive torques and/or zero net assistive torques during a physical therapy session. Accordingly, the disclosed systems provide a flexible platform for assisting motion of the joints of user in a number of different situations.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, in some embodiments, the disclosed devices may include one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, wirelessly connected devices, and pointing devices, such as mice, touch pads, and digital tablets. As another example, a device may receive input information through speech recognition or in other audible format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A device for assisting motion of a joint, the device comprising:
a motor;
a mandrel operatively coupled to the motor;
a tether operatively coupled to the mandrel and configured to wind onto the mandrel when the mandrel is rotated by the motor;
a guide configured to guide the tether as it is wound onto the mandrel, wherein the guide is configured to move in a direction substantially parallel to an axial length of the mandrel; and
at least one spring configured to bias the guide to a neutral position along the length of the mandrel.

2. The device of claim 1, wherein the guide includes an opening configured to receive the tether.

3. The device of claim 2, wherein the opening of the guide is made from a low friction material configured to minimize wear on the tether and a resistance to sliding motion between the tether and the guide.

4. The device of any of claims 1-3, wherein the at least one spring includes at least two springs disposed on opposing sides of the guide.

5. The device of any of claims 1-4, wherein the tether is a rope.

6. The device of claim 5, wherein the rope is a braided synthetic rope.

7. The device of any of claims 1-6, further comprising a tether enclosure enclosing the tether, wherein the tether enclosure extends and/or retracts as the tether is spooled on and/or off the mandrel.

8. The device of claim 7, wherein the tether enclosure is elastic.

9. The device of claim 8, wherein the tether enclosure is a textile.

10. The device of any of claims 1-9, wherein the guide is configured to wind the tether onto the mandrel in a single layer.

11. The device of any of claims 1-10, wherein at least the mandrel and the tether are selectively removable from the motor.

12. An actuator comprising:
a tether; and
a tether enclosure configured to at least partially enclose at least a portion of the tether, wherein the tether enclosure is configured to extend and/or retract when the tether extends and/or retracts from the actuator.

13. The actuator of claim 12, wherein the tether enclosure is elastic.

14. The actuator of claim 13, wherein the tether enclosure is a textile.

15. The actuator of any of claims 10-14, wherein a material of the tether enclosure is impermeable to a wet lubricant.

16. The actuator of any of claims 10-15, wherein the tether enclosure tapers from a first proximal end of the tether enclosure to a second distal end of the enclosure located proximate a distal portion of the tether.

17. A device for assisting motion of a joint, the device comprising:
a first anchor configured to be attached to a first body portion on a first side of the joint;
a second anchor configured to be attached to a second body portion on a second side of the joint;
an actuator operatively coupled to the first anchor and the second anchor; and
a support operatively coupled to the first anchor and the second anchor, wherein the support is configured to substantially maintain a position of the first anchor relative to the first body portion and/or a position of the second anchor relative to the second body portion.

18. The device of claim 17, wherein the support is configured to apply a torque to the joint when motion of the joint deforms the support.

19. The device of claim 18, wherein the torque applied to the joint by the support resists a torque applied to the joint by the actuator.

20. The device of any of claims 18-19, wherein the support is configured to substantially maintain a spacing of the first anchor and the second anchor.

21. The device of any of claims 18-20, wherein a relationship between the torque applied by the support and the deformation of the support is adjustable.

22. The device of any of claims 18-21, wherein the deformation of the support can be measured to calculate the torque applied by the support.

23. The device of any of claims 18-22, wherein the torque applied by the support can be measured with a torque sensor or calculated with measurements from one or more force sensors.

24. The device of any of claims 17-23, wherein at least a portion of the support is substantially aligned with an axial direction of a limb segment associated with the support.

25. A device for assisting motion of a joint, the device comprising:
a first anchor configured to be attached to a first body portion on a first side of the joint;
a second anchor configured to be attached to a second body portion on a second side of the joint;
a spring operatively coupled to the first anchor and the second anchor; and
an actuator operatively coupled to the first anchor and the second anchor, wherein actuating the actuator applies a torque about the joint that is resisted by a reaction torque from the spring.

26. The device of claim 25, wherein the spring comprises one or more selected from the group of a tension spring, a torsional spring, a leaf spring, and a curved elongated structure.

27. The device of either claim 25 or 26, wherein the joint comprises one or more selected from the group of an ankle, a knee, and a hip.

28. The device of any of claims 25-27, wherein the actuator is operatively coupled to a tether.

29. The device of claim 28, wherein the actuator is mounted on the first anchor.

30. The device of any of claims 25-29, wherein at least one of the first and second anchors comprises a flexible garment material configured to lay flat against at least one of the first and second body portions.

31. The device of any of claims 25-30, further comprising one or more sensors configured to sense the reaction torque from the spring.

32. The device of claim 31, wherein the torque applied by the actuator is adjusted based, at least in part, on the sensed reaction torque from the spring.

33. A method of assisting motion of a joint, the method comprising:
applying a first torque to the joint in a first direction with an actuator; and
applying a second torque to the joint in a second direction with a spring that is antagonistic to the first torque.

34. The method of claim 33, wherein applying the first torque to the joint in the first direction includes applying the first torque to the joint in the first direction such that the joint obtains a first configuration.

35. The method of either claim 33 or 34, further comprising removing the first torque.

36. The method of claim 34, further comprising continuing to apply the second torque with the spring such that the joint obtains a second configuration.

37. The method of any of claims 33-36, wherein the joint is an ankle, wherein one of the first and second directions is associated with plantarflexion, and wherein the other of the first and second directions is associated with dorsiflexion.

38. The method of any of claims 33-36, wherein the joint is a knee, wherein one of the first and second directions is associated with knee extension, and wherein the other of the first and second directions is associated with knee flexion.

39. The method of any of claims 33-36, wherein the joint is a hip, wherein one of the first and second directions is associated with hip extension, and wherein the other of the first and second directions is associated with hip flexion.

40. The method of any of claims 33-39, further comprising sensing one or more torques and/or forces applied to the joint and adjusting the first torque based at least partly on the one or more sensed torques and/or forces.

41. The method of claim 40, wherein sensing one or more torques and/or forces applied to the joint comprises sensing the second torque applied to the joint with the spring.
